(19) United States
(12) Patent Application Publication
(10) Pub. No.: US 2005/0031575 A1
(43) Pub. Date: Feb. 10, 2005

(54) Block copolymers

(76) Inventors: Stephen Brocchini, Welwyn Garden City (GB); Antony Godwin, Bristol (GB)

Correspondence Address: BELL & ASSOCIATES, 416 FUNSTON ST., SUITE 100, SAN FRANCISCO, CA, 94118, US

(21) Appl. No.: 10/500,536

(22) PCT Filed: Dec. 27, 2002
(86) PCT No.: PCT/GB02/05932

(30) Foreign Application Priority Data
Dec. 31, 2001 (GB) 0131112.5

(51) Int. Cl. A61K 31/785; C08K 5/07

(52) U.S. Cl. 424/78.27; 525/161

(57) ABSTRACT

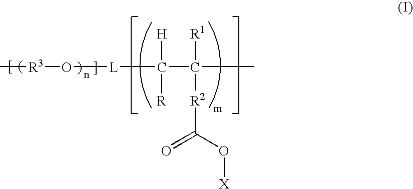

Novel block copolymers are described, together with the production therefrom of physiologically soluble polymer therapeutics. The block copolymers have the general formula (I) wherein R is selected from the group consisting of hydrogen, C₁-C₁₈ alkyl, C₂-C₁₈ alkenyl, C₇-C₁₈ aralkyl, C₇-C₁₈ alkaryl, C₆-C₁₈ aryl, carboxylic acid, C₂-C₁₈ alkoxycarbonyl, C²-C₁₈ alkaminocarbonyl, or any one of C₁-C₁₈ alkyl, C₂-C₁₈ alkenyl, C₇-C₁₈ aralkyl, C₇-C₁₈ alkaryl, C₆-C₁₈ aryl, C₂-C₁₈ alkoxycarbonyl and C₂-C₁₈ alkaminocarbonyl substituted with a heteroatom within, or attached to, the carbon backbone: R¹ is selected from the group consisting of hydrogen and C₁-C₆ alkyl groups; R² is a linking group; X is an electron withdrawing group; R³ is selected from the group consisting of C₁-C₁₅ alkylene, C₂-C₁₅ alkenylene, C₇-C₁₅ aralkylene, C₇-C₁₈ alarylene and C₆-C₁₈ arylene; l, is a divalent linker joining the of C₁-C₁₈ alkylene, C₂-C₁₈ alkylene, C₂-C₁₈ aralkylene, C₇-C₁₈ alarylene and C₆-C₁₈ arylene; l, is a divalent linker joining the blocks; and m and n are each an integer of greater than 1.

### FIELD OF THE INVENTION

The present invention is concerned with a class of block copolymers and the production therefrom of physiologically soluble polymer therapeutics, functionalised polymers, pharmaceutical compositions and materials.

### BACKGROUND OF THE INVENTION

Polymer Therapeutics are developed for biomedical applications requiring physiologically soluble polymers and include biologically active polymers, polymer-drug conjugates, polymer-protein conjugates, and other covalent constructs of polymer with bioactive molecules. An exemplary class of a polymer-drug conjugate is derived from copolymers of hydroxypropyl methacrylamide (HPMA) which have been extensively studied for the conjugation of cytotoxic drugs for cancer chemotherapy. An HPMA copolymer conjugated to doxorubicin, known as PK-1, is currently in Phase II evaluation in the UK. PK-1 displayed reduced toxicity compared to free doxorubicin in the Phase I studies. The maximum tolerated dose of PK-1 was 320 mg/m² which is 4-5 times higher than the usual clinical dose of free doxorubicin.

The polymers used to develop Polymer Therapeutics may also be separately developed for other biomedical applications where the polymer conjugate is developed (e.g. as a block copolymer) to form aggregates such as polymeric micelles and complexes. The polymers used to develop Polymer Therapeutics may also be separately developed for other biomedical applications that require the polymer be used as a material rather than as a physiologically soluble molecule. Thus, drug release matrices (including microspheres and nanoparticles), hydrogels (including injectable gels and viscious solutions) and hybrid systems (e.g. liposomes with conjugated poly(ethylene glycol) (PEG) on the outer surface) and devices (including rods, pellets, capsules, films, gels) can be fabricated for tissue or site specific drug delivery. Polymers are also clinically widely used as excipients in drug formulation. Within these three broad application areas: (1) physiologically soluble molecules, (2) materials and (3) excipients, biomedical polymers provide a broad technology platform for optimising the efficacy of a therapeutic bioactive agent.

Therapeutic bioactive agents which can be covalently conjugated to a polymer include a drug, peptide and protein. Such conjugation to a soluble, biocompatible polymer can result in improved efficacy of the therapeutic agent. Compared to the free, unconjugated bioactive agent, therapeutic polymeric conjugates can exhibit this improvement in efficacy for the following main reasons: (1) altered biodistribution, (2) prolonged circulation, (3) release of the bioactive in the proteolytic and acidic environment of the secondary lysosome after cellular uptake of the conjugate by pinocytosis and (4) more favourable physicochemical properties imparted to the drug due to the characteristics of large molecules (e.g. increased drug solubility in biological fluids).

Co-block copolymers, comprising hydrophilic and hydrophobic blocks, form polymeric micelles in solution [Kataoka, Kwon, Yokoyama, Okano and is Sakurai J. Cont.Rel. 1993, 24, 119, Gros, Ringsdorf and Schupp Angew.Chemie Int. Ed. Eng. 1981, 20, 301, Kwon, Yokoyama, Okano, Sakurai and Kataoka Pharm. Res. 1993, 10, 970, Kwon and Kataoka Adv. Drug. Del. Rev. 1995, 16, 295, Kwon and Okano Adv. Drug Del. Rev. 1996, 21, 107, Yokoyama Crit.Rev.Therap.Drug Carrier Systems 1992, 9, 213] and self-assembling micellar delivery systems are receiving increasing attention [Alakhov and Kabanov Exp. Opin. Invest. Drugs 1998, 7, 1453, Calibresi and Chabner The Pharmacological Basis of Therapeutics 1996, 1225, Kabanov and Alakhov J. Cont.Rel. 1994, 28, 15, Yokayama, Okano, Sakurai and Kataoka J. Cont. Rel. 1994, 32, 269]. A significant advantage of these systems is the ability to design higher molecular weight micellar aggregates that will display prolonged circulation times that can maximise tumour capture by the EPR effect. Upon micelle disassociation, the individual block copolymer molecules are safely excreted, and as long as they are of low enough molecular weight these polymers can be non-biodegradable. For example, poly(ethylene glycol-aspartate) block copolymer doxorubicin conjugates form micelles ranging in size from 20-60 nm that accumulate in solid tumours and exhibit antitumour activity [Kataoka, Kwon, Yokoyama, Okano and Sakurai J. Cont.Rel. 1993, 24, 119, Kwon, Yokoyama, Okano, Sakurai and Kataoka Pharm. Res. 1993, 10, 970, Kwon and Kataoka Adv. Drug.Del.Rev. 1995, 16, 295, Kataoka Controlled drug delivery—challenges and strategies 1997, 49, Yokoyama, Okano, Sakurai, Ekimoto, Shibazak and Kataoka Cancer Res. 1991, 51, 3229]. The doxoribicin is conjugated by its free amine directly to either the a- or b-pendent carboxylates in the poly(aspartic acid) block. Frequently physical entrapment of drug has accompanied conjugation [Yokoyama, Fukushima, Uehara, Okamoto, Kataoka, Sakurai and Okano J. Cont. Rel. 1998, 50, 79] and with stable block copolymer micelles, drug entrapment has become a viable strategy to deliver cytotoxic drugs to tumours [Alakhov and Kabanov Exp. Opin. Invest. Drugs 1998, 7, 1453, Yokoyama, Fukushima, Uehara, Okamoto, Kataoka, Sakurai and Okano J. Cont. Rel. 1998, 50, 79, Batrakova, Dorodnych, Klinskii, Kliushnenkova, Shemchukova, Goncharova, Ajakov, Alakhov and Kabanov Br. J. Cancer 1996, 74, 1545, Venne, Li, Mandeville, Kabanov and Alakhov Cancer Res. 1996, 56, 3626, Inoue, Chen, Nakamae and Hoffman J. Cont. Rel. 1998, 51, 221].

Poly(acrylic acid), poly(methaacrylic acid) and poly(ethylene glycol) based excipients are widely used to modify adhesion, swelling and pH dependent properties of tablets and pharmaceutical formulations. Incremental variation in the stoichiometry of the conjugation reactions of functionalised amines provide libraries of narrow MWD candidate polymers. This will make it possible to optimise the materials properties that include thermal properties, crystallisation, adhesion, swelling, coating and pH dependent conformation either independently or collectively. Of these many materials properties, controlling the rate of crystallisation processes tends to influence the stability, solubility and activity of chemically and biologically sensitive drugs (e.g. proteins). Hence, functionalised excipients designed to slow crystallisation processes and maintain unstable amorphous morphologies of pharmaceutical formulations (i.e. blends) may find wide use.

Additionally copolymeric excipients [Kabanov, Alakov and Batrakova PCT-WO 99/39731 1999, 80 pages, Galakatos, Langer and Putnam PCT-WO059627 2000, 46 pages, Alakhov, Klinski, Li, Piertrzynski, Venne, Batrakova, Bronitch and Kabanov Colloids Surf., B. 1999, 16, 114, Lemieux, Guerin, Paradis, Proulz, Chistyakova, Kabanov and Alakhov Gene Ther. 2000, 7, 986] and nanoscopic particles [Boal, Ilhan, DeRouchey, Thurn-Albrecht, Russell and Rotello Nature 2000, 404, 746] have been examined. Many excipients that are generally recognised as safe have been evaluated to determine a multitude of trends that can be matched to the physicochemical properties of the pharmcologically active compounds. A doxorubicin formulation using a combination of two pluronics has shown this formulation may have broader efficacy than current clinical formulations of doxorubicin [Alakhov and Kabanov Exp. Opin. Invest. Drugs 1998, 7, 1453]. Since coblock polymers form aggregated micellar structures these may be potentially developed into novel formulations for the oral administration of bioactive agents.

Polymer-drug conjugates tend to be non-uniform with respect to molecular weight of the polymer and the location and number of conjugating pendent chains along the polymer mainchain. Polymer therapeutics must be rigorously characterised with respect to their molecular weight and polydispersity since biodistribution and pharmacological activity are known to be molecular weight-dependent. For example, blood circulation half-life [Cartlidge, Duncan, Lloyd, Kopeckova-Rejmanova and Kopecek J Con. Rel. 1986, 4, 253], renal clearance, deposition in organs [Sprincl, Exner, Sterba and Kopecek J. Biomed. Mater. Res. 1976, 10, 953], rates of endocytic uptake [Duncan, Pratten, Cable, Ringsdorf and Lloyd Biochem. J. 1981, 196, 49, Cartlidge, Duncan, Lloyd, Rejmanova and Kopecek J. Cont. Rel. 1986, 3, 55] and biological activity can depend on polymer molecular weight characteristics [Kaplan Anionic Polymeric Drugs 1980, 227, Ottenbrite, Regelson, Kaplan, Carchman, Morahan and Munson Polymeric Drugs 1978, 263, Butler Anionic Polymeric Drugs 1980, 49, Muck, Rolly and Burg Makromol. Chem. 1977, 178, 2773, Muck, Christ and Keller Makromol. Chem. 1977, 178, 2785, Seymour J. Bioact. Compat. Polymers 1991, 6, 178]. While HPMA copolymers currently undergoing clinical evaluation exhibit increased efficacy and a considerable amount of the biological rationale for polymer-drug conjugates has been elucidated, the fact is these therapeutic compounds exist as broad statistical distributions in respect to molecular weight and structure of conjugation pendent chains. This is problematic from a regulatory standpoint, especially for chronic conditions. For example, it would be difficult to ascertain if long term effects were due to low or high molecular weight species in a polydisperse therapeutic conjugate.

Currently many candidate copolymer-drug conjugates are prepared by a reaction on a polymer or a polymer analogous reaction of a low molecular weight drug and an active ester polymeric precursor with a small number of reactive repeat units [Kopecek and Bazilova Eur. Polymer J. 1973, 9, 7, Strohalm and Kopecek Angew. Makromol. Chem. 1978, 70, 109, Rejmanova, Labsky and Kopecek Makromol. Chem. 1977, 178, 2159, Kopecek Makromol. Chem. 1977, 178, 2169, Rihova, Ulbrich, Strohalm, Vetvicka, Bilej, Duncan and Kopecek Biomaterials 1989, 10, 335, Kopecek J. Cont. Rel. 1990, 11, 279].

Many conjugates have been prepared by the polymer analogous reaction however the competitive hydrolysis of the p-nitrophenol ester actually produces conjugates that have pendent chains terminated with either drug, carboxylate, or aminopropanol [Configliacchi, Razzano, Rizzo and Vigevani J. Pharm. Biomed. Analysis 1996, 15, 123, Pinciroli, Rizo, Angelucci, Tato and Vigevani Magn. Reson. Chem. 1997, 35, 2]. The free radical precipitation polymerisation gives the active ester polymeric precursor with a small number of reactive repeat units as a random copolymer typically with a polydisersity ranging from 1.3-2.5 and above depending on the pendent chain. Also such precipitation polymerisation strategies only give polymeric precursors with a small range of molecular weights. Incorporation of different amounts monomers with different pendent chains requires that the polymerisation conditions be optimised to obtain reproducible molecular weights under the renal threshold. In principal, it is possible to alter drug loading by varying its stoichiometry during conjugation but the final polymeric conjugate will contain mixtures of the unreacted pendent chains with out drug that are statistically distributed over a broad molecular weight distribution. Lysosomal degradation of non-drug conjugated pendent chains will compete with degradation of the drug conjugated pendent chains. This competition complicates the pharmacology and pharmacokinetics for polymer-drug conjugates. From the viewpoint of drug regulatory authorities, this strategy for preparing conjugates result in final polymer-drug conjugates that are extremely varied in structure and thus difficult to regulate as a medicinal agent.

In addition to regulatory issues and as mentioned above, structural heterogeneity will influence the pharmacology and pharmacokinetics of therapeutic conjugates. For example, the rate of drug release from a given polymer chain can vary according to the structure of the pendent chain and drug [Duncan Anti-Cancer Drugs 1992, 3, 175, Duncan, Seymour, Ulbrich, Spreafico, Grandi, Ripamonti, Farao and Suarato Eur. J. Cancer 1991, 27, S52]. Rates of release are also influenced by the amount (i.e. loading) and location along the polymer mainchain of the conjugated drug. As greater amounts of hydrophobic drug are conjugated onto a soluble hydrophilic polymer, the possibility increases to form unimolecular polymeric micelles which may hinder access of the lysosomal enzymes to degrade the linker and release the conjugated drug [Ulbrich, Konak, Tuzar and Kopecek Makromol. Chem. 1987, 188, 1261]. Hydrophobic drugs conjugated to hydrophilic polymers can result in a lower critical solution temperature (LCST) where phase separation occurs and the conjugate becomes insoluble. Also, as a drug is released from a polymer-drug conjugate, it would be expected that changes in polymer conformation will occur that might lead to differences in drug release rate with time [Pitt, Wertheim, Wang and Shah Macromol. Symp. 1997, 123, 225, Shah, Werthim, Wang and Pitt J. Cont. Rel. 1997, 45, 95] and therefore influence pharmacological properties. The extent and location of drug loading and its influence on polymer solution properties is an important, and yet poorly understood phenomenon that has a fundamental effect on the in vivo properties of therapeutic polymer-conjugates.

It is evident that as mixtures of structures, the polydisperse and randomly conjugated polymer-drug conjugates which are being studied are not optimal. While a significant amount is known about the biological rationale for the development of the polymer therapeutics, there is less known about the chemical rationale. Three broad needs related to chemical structure worthy of systematic study have been identified to extend the use of soluble addition polymers in medicine: (1) preparation of relevant polymers with narrow molecular weight and pendent chain distribution, (2) use of suitable conjugation strategies that minimise competitive reactions and (3) controlled placement of conjugating pendent chains along the polymer mainchain (e.g. preparation of block copolymers).

To address these chemical limitations for preparing therapeutic conjugates, WO 01/18080 describes the production of low molecular weight distribution homo-and copolymers, including block copolymers, having a polydispersity less than 1.4. Polymerisation was carried out by controlled radical polymerisation processes to give narrow molecular weight polymeric precursors that are used as precursor polymers to prepare a wide range of metha- and acrylamide homo-and copolymers. Only a few metha- and acrylamide homo-and copolymers with narrow molecular distribution can be prepared directly from polymerization. These are used in the production of polymer drug conjugates having desirable biological profiles.

### SUMMARY OF THE INVENTION

In a first aspect of the present invention, there is provided a block copolymer comprising the unit (I) 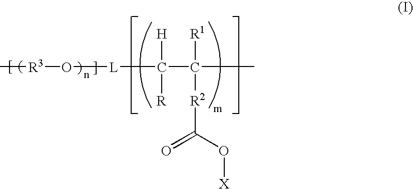
wherein R is selected from the group consisting of hydrogen, C₁-C₁₈ alkyl, C₂-C₁₈ alkenyl, C₇-C₁₈ aralkyl, C₇-C₁₈ alkaryl, C₆-C₁₋₈ aryl, carboxylic acid, C₂-C₁₈ alkoxycarbonyl, C₂-C₁₈ alkaminocarbonyl, or any one of C₁-C₁₋₈ alkyl, C₂-C₁₈, alkenyl, C₇-C₁₈ aralkyl, C₇-C₁₈ alkaryl, C₆-C₁₈ aryl, C₂-C₁₈ alkoxycarbonyl and C₂-C₁₈ alkaminocarbonyl substituted with a heteroatom within, or attached to, the carbon backbone; R¹ is selected from the group consisting of hydrogen and C₁-C₆ alkyl groups; R² is a linking group; X is an electron withdrawing group; R³ is selected from the group consisting of C₁-C₁₈ alkylene, C₂-C₁₈ alkenylene, C₇-C₁₈ aralkylene, C₇-C₁₈ alkarylene and C₆-C₁₈ arylene; L is a divalent linker joining the blocks; and m and n are each an integer of greater than 1.

The copolymer (I) is an A-B type block copolymer. It may be an A-B-A or A-B-C type block copolymer. The substructures defined in the square parentheses are the blocks. Preferably, m and n are integers of 5 to 300, more preferably 10 to 200, most preferably 25 to 150.

Preferably the block copolymer has a polydispersity of less than 1.4, preferably less than 1.2 and a molecular weight (Mw) of less than 100,000. Preferably (I) is water soluble.

X is preferably individually selected for each block and may be the same or different.

The electron withdrawing group X is preferably a carboxylate activating group, and is preferably selected from the group consisting of N-succinimidyl, pentachlorophenyl, pentafluorophenyl, para-nitrophenyl, dinitrophenyl, N-phthalimido, norbornyl, cyanomethyl, N-pyridyl, N-trichlorotriazine, 5-chloroquinilino, and N-imidazole. Preferably X is an N-succinimidyl or imidazole moiety.

Preferably R is selected from the group consisting of hydrogen, C₁-C₆ alkyl, C₁-C₆ alkenyl, C₁-C₆ aralkyl and C₁-C₆ alkaryl, C₂-C₈ alkoxycarbonyl, C₂-C₈ alkaminocarbonyl. Most preferably R is selected from hydrogen and methyl.

Preferably R¹ is selected from the group consisting of hydrogen, methyl, ethyl, propyl, butyl, pentyl or isomers thereof. Most preferably R¹ is selected from hydrogen and methyl.

Preferably R² is selected from a bond or contains at least 1 carbon atom or at least 1 heteroatom.

Where R² is not a bond, R² is connected to CR¹ via a divalent group, preferably comprising a carbonyl, C₁-C₁₈ alkylene and/or C₆-C₁₈ arylene group which may be substituted with 1 or more heteroatoms. Preferably R² comprises a group selected from the group consisting of C₁-C₆ alkylene, C₆-C₁₂ arylene, C₁-C₁₂ oxyalkylene and carbonyl-C₁-C₆ alkylene. Where R² comprises an alkylene group, it can be branched, linear or cyclical, substituted or unsubstituted with one or more alkyl groups, and is preferably methylene, 1,2-ethylene, 1,3-propylene, hexylene or octylene. Where R² comprises an arylene group, preferably it is benzylene, tolylene or xylylene.

Preferably the groups R³, which may be the same or different, are selected from the group consisting of C₁-C₈ alkylene groups, preferably 1,2-alkylene, and C₆-C₁₂ arylene groups, most preferably methylene, ethylene, 1,2-propylene and 1,3-propylene. Preferably all groups R³ are the same, most preferably all are 1,2-ethylene or 1,2-propylene.

L preferably comprises a C₁-C₁₈ alkylene or C₆-C₁₈ arylene group which may be substituted and/or interrupted with 1 or more heteroatoms. Preferably L comprises a group selected from the group consisting of C₁-C₆ alkylene, C₆-C₁₂ arylene, C₁-C₁₂ oxyalkylene and C₁-C₆ acyl. Where L comprises an alkylene group, it can be branched, linear or cyclical, substituted or unsubstituted with one or more alkyl groups, and is preferably methylene, 1,2-ethylene, 1,2-propylene, 1,3-propylene, ^{tert}butylene, ^{sec}butylene, hexylene or octylene. Where L comprises an arylene group, it is preferably benzylene, tolylene or xylylene. Most preferably L comprises a —COR^{a} group, wherein R⁸ is selected from the group consisting of C₁-C₆ alkylene or C₆-C₁₂ arylene, preferably methylene, 1,2-ethylene, 1,2-propylene, 1,3-propylene, ^{tert}butylene and ^{sec}butylene.

The block copolymer of the present invention may incorporate other polymeric, oligomeric or monomeric blocks. For example, further polymeric blocks incorporated in the polymer may comprise acrylic polymers, alkylene polymers, urethane polymers, amide polymers, polypeptides, polysaccharides and ester polymers.

The molecular weight of the block copolymer should ideally be less than 100,000, preferably less than 50,000 where the block copolymer is to be used as a physiologically soluble block copolymer (in order that the renal threshold is not exceeded, ie to ensure that the polymer is cleared from the kidney glomerulus). Preferably the molecular weight of the block copolymer is in the range of 4000-50,000, more preferably 25,000-40,000.

A further preferred aspect of the present invention provides a block copolymer comprising the structure (II) 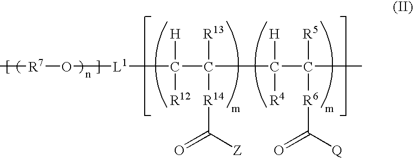
wherein R⁴ is selected from the group consisting of hydrogen, C₁-C₁₈ alkyl, C₂-C₁₈ alkenyl, C₇-C₁₈ aralkyl, C₇-C₁₈ alkaryl, C₆-C₁₈ aryl, carboxylic acid, C₂-C₁₈ alkoxycarbonyl, C₂-C₁₈ alkaminocarbonyl, or any one of C₁-C₁₈ alkyl, C₂-C₁₈ alkenyl, C₇-C₁₈ aralkyl, C₇-C₁₈ alkaryl, C₆-C₁₈ aryl, C₂-C₁₈ alkoxycarbonyl, and C₂-C₁₈ alkaminocarbonyl substituted with a heteroatom within, or attached to, the carbon backbone; R⁵ is selected from the group consisting of hydrogen and C₁-C₈ alkyl groups; R⁶ is a linking group; Q is a solubilising group selected from the group consisting of hydroxyl, C₁-C₁₂ alkyl, C₂-C₁₂ alkenyl, C₇-C₁₂ aralkyl, C₇-C₁₂ alkaryl, C₁-C₁₂ alkoxy, C₁-C₁₂ hydroxyalkyl, C₁-C₁₂ alkylamino, C₁-C₁₂ hydroxyalkylamino, or any of C₁-C₁₂ alkyl, C₂-C₁₂ alkenyl, C₇-C₁₂ aralkyl, C₇-C₁₂ alkaryl, C₁-C₁₂ alkoxy, C₁-C₁₂ hydroxyalkyl, C₁-C₁₂ alkylamino, C₁-C₁₂ alkylamino substituted with an amine, hydroxyl, carbonyl or thiol group; R⁷ is selected from the group consisting of C₁-C₁₈ alkylene, C₂-C₁₈ alkenylene, C₇-C₁₈ aralkylene, C₇-C₁₈ alkarylene and C₆-C₁₈ arylene; n, m and p are each an integer of greater than 1; R¹² is selected from the group consisting of hydrogen, C₁-C₁₈ alkyl, C₂-C₁₈ alkenyl, C₇-C₁₈ aralkyl, C₇-C₁₈ alkaryl, C₆-C₁₈ aryl, carboxylic acid, C₂-C₁₈ alkoxycarbonyl, C₂-C₁₈ alkaminocarbonyl, or any one of C₁-C₁₈ alkyl, C₂-C₁₈ alkenyl, C₇-C₁₈ aralkyl, C₇-C₁₈ alkaryl, C₈-C₁₈ aryl, C₂-C₁₈ alkoxycarbonyl, and C₂-C₁₈ alkaminocarbonyl substituted with a heteroatom within, or attached to, the carbon backbone; R¹³ is selected from the group consisting of hydrogen and C₁-C₆ alkyl groups; R¹⁴ is a linking group; L¹ is a divalent linker joining the blocks; Z is a pendent group selected from the group consisting of OM_{1/d}^{d+}, NR⁸R⁹, SR¹⁰, OR¹¹ and OX, wherein X is defined above, M is a metal ion and d is an integer of 1 or 2, R⁸ comprises an alkyl group, preferably an aminoacyl substituted alkyl group, more preferably oligopeptidyl group; R⁹ is selected from hydrogen, C₁-C₁₈ alkyl, C₂-C₁₈ alkenyl, C₇-C₁₈ aralkyl, C₇-C₁₈ alkaryl; R¹⁰ and R¹¹ comprise a group which is individually selected from the group consisting of hydrogen, C₁-C₁₂ alkyl, C₂-C₁₂ alkenyl, C₇-C₁₂ aralkyl, C₇-C₁₂ alkaryl and C₁-C₁₂ hydroxyalkyl, and may contain one or more cleavable bonds and may comprise a bioactive agent.

M is preferably a sodium or potassium ion.

Z is preferably individually selected for each block and may be the same or different. Thus, different pendent groups may be attached to different blocks.

Q is preferably individually selected for each block and may be the same or different.

Preferably the block copolymer of this aspect of the invention has a polydispersity of less than 1.4, preferably less than 1.2 and a molecular weight (Mw) of less than 100,000. Preferably (II) is water soluble. The molecular weight of the block copolymer is preferably less than 50,000, more preferably in the range of 4000-50,000, most preferably 25,000-40,000.

Preferably R⁴ is selected from the group consisting of hydrogen, C₁-C₆ alkyl, C₁-C₆ alkenyl, C₁-C₆ aralkyl and C₁-C₆ alkaryl, C₂-C₈ alkoxycarbonyl, C₂-C₈ alkaminocarbonyl. Most preferably R⁴ is selected from hydrogen and methyl.

Preferably R⁵ is selected from the group consisting of hydrogen, methyl, ethyl, propyl, butyl, pentyl or isomers thereof. Most preferably R¹ is selected from hydrogen and methyl.

Preferably R⁶ is selected from a bond or contains at least 1 carbon atom or at least 1 heteroatom.

Where R⁶ is not a bond, R⁶ is connected to CR⁵ via a divalent group, preferably comprising a carbonyl, C₁-C₁₈ alkylene and/or C₆-C₁₈ arylene group which may be substituted with 1 or more heteroatoms. More preferably R⁶ comprises a group selected from the group consisting of C₁-C₆ alkylene, C₆-C₁₂ arylene, C₁-C₁₂ oxyalkylene and carbonyl-C₁-C₆ alkylene. Where R⁶ comprises an alkylene group, it can be branched, linear or cyclical, substituted or unsubstituted with one or more alkyl groups, and is preferably methylene, 1,2-ethylene, 1,2-propylene 1,3-propylene, ^{tert}butylene, ^{sec}butylene, hexylene or octylene. Where R⁵ comprises an arylene group, it is preferably benzylene, tolylene or xylylene.

Preferably the groups R⁷, which may be the same or different, are selected from the group consisting of C₁-C₈ alkylene groups, preferably 1,2-alkylene, and C₆-C₁₂ arylene groups, most preferably methylene, ethylene, 1,2-propylene and 1,3-propylene. Preferably all groups R⁷ are the same, most preferably all are 1,2-ethylene or 1,2-propylene.

Z may comprise a protecting group, ie be a group OX, where X is defined above.

Z may comprise a peptidic group. Preferably Z comprises one or more aminoacyl groups, preferably 2-6 aminoacyl groups, most preferably 4 aminoacyl groups. In a particularly preferred embodiment group Z comprises a glycine-leucine-phenylalanine-glycine linker. The aminoacyl linker is most preferably a peptide linker capable of being cleaved by preselected cellular enzymes, for instance, those found in the liposomes found in cancerous cells.

In a further aspect, group Z comprises a cis-aconityl group. This group is designed to remain stable in plasma at neutral pH (7.4), but degrade intracellularly by hydrolysis in the more acidic environment of the endosome or liposome (˜pH 5.5-6.5). This is particularly advantageous for the treatment of cancer as there are marked improvements in therapeutic efficacy and site specific passive capture through the enhanced permeability and retention (EPR) effect. The EPR effect results from enhanced permeability of macromolecules or small particles within the tumour neovasculature due to leakiness of its discontinuous endothelium. In addition to the tumour angiogenesis (hypervasculature) and irregular and incompleteness of vascular networks, the attendant lack of lymphatic drainage promotes accumulation of macromolecules that extravasate. This effect is observed in many solid tumours for macromolecular agents and lipids. Thus, increased accumulation in such tumours leads to a targeted delivery of a group Z incorporating a bioactive agent (as discussed below) and a cis-aconityl group.

The pendent chain Z may additionally comprise a ligand or bioactive agent. The ligand may be any ligand which is capable of polyvalent interactions. Preferred bioactive agents are anti-cancer agents such as doxorubicin, daunomycin and paclitaxel. The bioactive agent is preferably joined to R¹⁴CO via a peptidic linker.

L¹ preferably comprises a C₁-C₁₈ alkylene and/or C₆-C₁₈ arylene group which may be substituted and/or interrupted with 1 or more heteroatoms. Preferably L¹ comprises a group selected from the group consisting of C₁-C₆ alkylene, C₆-C₁₂ arylene, C₁-C₁₂ oxyalkylene and C₁-C₆ acyl. Where L¹ comprises an alkylene group, it can be branched, linear or cyclical, substituted or unsubstituted with one or more alkyl groups, and is preferably methylene, 1,2-ethylene, 1,2-propylene 1,3-propylene, ^{tert}butylene, ^{sec}butylene, hexylene or octylene. Where L¹ comprises an arylene group, preferably itis benzylene, tolylene or xylylene. Most preferably L¹ comprises a —COR^{a} group, wherein R^{a} is defined above with regard to (I).

It should be understood that the graphical representation of (II) above is not intended to limit the order within the blocks bordered by parentheses, ie. the substructure containing Q may be adjacent to the block containing R⁷.

Preferably, p is an integer of 1 to 500, more preferably 20 to 200.

Preferred definitions of R¹², R¹³ and R¹⁴ are the same groups as R⁴, R⁵ and R⁶ respectively.

Preferably Q comprises an amine group attached to the R⁶CO carbonyl carbon, preferably a C₁-C₁₂ hydroxyalkylamino group, most preferably a 2-hydroxypropylamino group. This group is designed to be a solubilising group for the block copolymer in aqueous solutions. Generally the block copolymer of the present invention is a water soluble polyacrylamide/polyalkyleneglycol block copolymer, preferably a polymethacrylamide or polyethacrylamide/polyethyleneglycol block copolymer.

In a further aspect, the present invention provides a process for the production of a block copolymer, comprising the polymerisation of ethylenically unsaturated monomers including a compound (III) 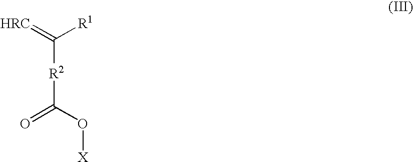
wherein R is selected from the group consisting of hydrogen, C₁-C₁₈ alkyl, C₂-C₁₈ alkenyl, C₇-C₁₈ aralkyl, C₇-C₁₈ alkaryl, C₆-C₁₈ aryl, carboxylic acid, C₂-C₁₈ alkoxycarbonyl, C₂-C₁₈ alkaminocarbonyl, or any one of C₁-C₁₈ alkyl, C₂-C₁₈ alkenyl, C₇-C₁₈ aralkyl, C₇-C₁₈ alkaryl, C₆-C₁₈ aryl, C₂-C₁₈ alkoxycarbonyl, and C₂-C₁₈ alkaminocarbonyl substituted with a heteroatom within, or attached to, the carbon backbone; R¹ is selected from the group consisting of hydrogen and C₁-C₆ alkyl groups; R² is a linking group; X is an electron withdrawing group; in the presence of an initiator compound (IV)
R¹⁵(R³O)ₙ—Y (IV)
wherein n is an integer of 1 or more and Y is a radical initiating group; R³ is selected from the group consisting of C₁-C₁₈ alkylene, C₂-C₁₈ alkenylene, C₇-C₁₈ aralkylene, C₇-C₁₈ alkarylene and C₆-C₁₈ arylene; R¹⁵ comprises a group selected from the group consisting of hydrogen, C₁-C₁₈ alkyl, C₂-C₁₈ alkenyl, C₇-C₁₈ aralkyl, C₇-C₁₈ alkaryl and C₆-C₁₈ aryl, C₁-C₁₈ alkoxy, C₂-C₁₈ alkeneyloxy, C₇-C₁₈ aralkoxy, C₇-C₁₈ alkaryloxy, C₆-C₁₈ aryloxy and —O—Y; to produce a block copolymer comprising the unit (V) 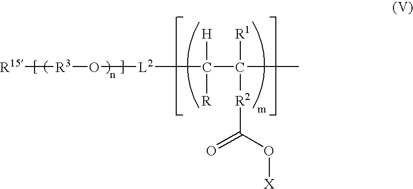
wherein m and n are as defined above and L² is a divalent linking group derived from Y and R^{15′} is R¹⁵, or where R¹⁵ is —O—Y, R¹⁵ is

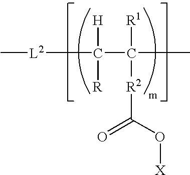

Preferred examples of the definitions of X, R, R¹, R² and R³ are as defined above in respect of (I).

Y preferably comprises a halogen substituted C₁-C₁₈ alkyl or C₆-C₁₈ aryl group, preferably bromine or chlorine substituted. Preferably Y comprises a group selected from the group consisting of C₁-C₆ alkyl, C₆-C₁₂ aryl, C₁-C₁₂ oxyalkyl and C₁-C₆ acyl substituted with 1 or more halogen atoms. Where Y comprises an alkyl group, it can be branched, linear or cyclical, substituted or unsubstituted with one or more alkyl groups, and is preferably methyl, ethyl, propyl, ^{tert}butyl, ^{sec}butyl, hexyl or octyl. Where Y comprises an aryl group, it is preferably benzyl, tolyl or xylyl. Most preferably Y comprises a —COR^{y} group, wherein R^{y} is selected from the group consisting of halogen substituted C₁-C₆ alkyl or C₆-C₁₂ aryl, preferably methyl, ethyl, propyl, ^{tert}butyl and ^{sec}butyl. Most preferably Y is —CO^{tert}butylbromide.

L² is preferably derived from Y, ie, the product of the radical reaction with monomer, and is a C₁-C₁₈ alkylene and/or C₆-C₁₈ arylene group which may be substituted with 1 or more heteroatoms. Preferably L² comprises a group selected from the group consisting of C₁-C₆ alkylene, C₆-C₁₂ arylene, C₁-C₁₂ oxyalkylene and carbonyl-C₁-C₆ alkylene. Where L² comprises an alkylene group, it can be branched, linear or cyclical, substituted or unsubstituted with one or more alkyl groups, and is preferably methylene, 1,2-ethylene, 1,2-propylene, 1,3-propylene, ^{tert}butylene, ^{sec}butylene, hexylene or octylene. Where L² is an arylene group, preferably it is benzylene, tolylene or xylylene. Most preferably L² is a —COR^{a} group, wherein R^{a} is selected from the group consisting of C₁-C₆ alkylene or C₆-C₁₂ arylene, preferably methylene, 1,2-ethylene, 1,2-propylene, 1,3-propylene, ^{tert}butylene and ^{sec}butylene.

R¹⁵ is preferably selected from hydrogen, C₁-C₆ alkyl, C₁-C₆ alkoxy, C₂-C₁₀ alkenyl, C₇-C₁₀ aralkyl, C₇-C₁₀ alkaryl and C₆-C₁₀ aryl and —O—Y, more preferably is methoxy or —O—Y (which will produce an A-B-A type block copolymer).

Preferably the process is a controlled radical polymerization.

Where the polymerization is carried out by atom transfer radical polymerization, the polymerisation is preferably carried out in the presence of a polymerisation mediator comprising a Cu(I) complex. Such complexes are usually Cu(I)Br complexes, complexed by a chelating ligand. Typical mediators are Cu(I)Br (Bipy)₂, Cu(I)Br (Bipy), Cu(I)Br (Pentamethyl diethylene), Cu(I)Br[methyl₆ tris(2-aminoethyl)amine] and Cu(I)Br(N,N,N′,N″,N″-pentamethyldiethylenetriamine).

The ethylenically unsaturated monomers may include comonomers copolymerisable with the monomer of the formula (III).

The reaction should take place in the presence of a suitable solvent.

Such solvents are generally aprotic solvents, for example tetrahydrofuran, acetonitrile, dimethylformamide, acetone, dimethylsulphoxide, ethyl acetate, methylformamide, ethylene carbonate and sulpholane and mixtures thereof. Alternatively, water may be used. Particularly preferred solvents are dimethylsulphoxide, ethylene carbonate, tetrahydrofuran, and dimethylformamide and mixtures thereof.

Preferably (V) may be reacted further with a reagent HR^{x}, wherein R^{x} is selected from the group consisting of NR¹⁹R²⁰, SR²¹ and OR²², wherein R¹⁹ is or comprises a linker group, preferably a substituted alkyl group, more preferably a peptidic group; R²⁰ is selected from hydrogen, C₁-C₁₈ alkyl, C₂-C₁₈ alkenyl, C₇-C₁₈ aralkyl, C₇-C₁₈ alkaryl, C₆-C₁₈ aryl; R²¹ and R²² are selected from the group consisting of hydrogen, C₁-C₁₂ alkyl, Cd-C₁₋₂ alkenyl, C₁-C₁₂ aralkyl, C₁-C₁₂ alkaryl, C₁-C₁₂ alkoxy and C₁-C₁₂ hydroxyalkyl, any of which may comprise a bioactive agent substituent and/or may contain one or more cleavable bonds, to form a derivatised block copolymer having the structure (VI) 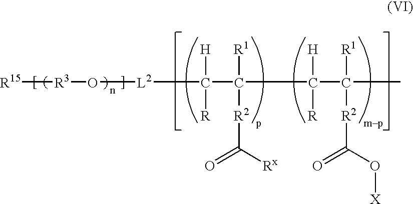
wherein 1≦p≦m.

Preferably, p is an integer of 1 to 200, more preferably 1 to 10. Preferably HR^{x} is H₂NR^{z}.

HR^{x} is generally a nucleophilic reagent capable of displacing X—O, to form a covalent bond with the acyl group attached to R². Most preferably R^{z} comprises a cleavable bond such as a aminoacyl linker or a cis-aconityl linker as described hereinbefore. Generally R^{z} comprises a bioactive agent substituent, which may have been attached prior to reaction with (V).

Subsequent to the production of a block copolymer having the structure (VI), an additional step of quenching the block copolymer may take place. This involves reacting the previously unreacted groups COOX with a quenching group. This group preferably comprises an amine moiety and is generally selected to be a solubilising or solubility modifying group for the block copolymer. Such a quenching compound is, for example a hydrophilic reagent, for example, hydroxypropylamine. Different types of quenching groups may be employed in the same polymer.

Alternatively (I) may be reacted with a reagent HR^{x} as defined above, to form a compound (VII) 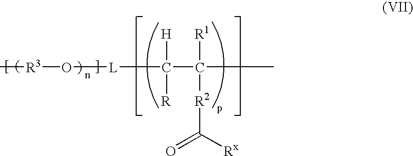
wherein R, R¹, R², R³, R^{x}, n, p and L are as defined above. This compound may be further reacted with a quenching group. These groups react with any unreacted groups COOX. This group preferably comprises an amine moiety and is generally selected to be a solubilising or solubility modifying group for the block copolymer. Such a quenching compound is, for example a hydrophilic reagent, for example, hydroxypropylamine.

In a further aspect, the present invention provides a process for the production of a block copolymer, comprising the steps of (1) polymerising ethylenically unsaturated monomers comprising a compound (VII) 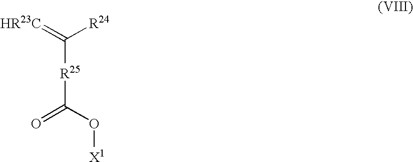
wherein R²³ is selected from the group consisting of hydrogen, C₁-C₁₈ alkyl, C₂-C₁₈ alkenyl, C₇-C₁₈ aralkyl, C₇-C₁₈ alkaryl, C₆-C₁₈ aryl, carboxylic acid, C₂-C₁₈ alkoxycarbonyl, C₂-C₁₈ alkaminocarbonyl, or any one of C₁-C₁₈ alkyl, C₂-C₁₈ alkenyl, C₇-C₁₈ aralkyl, C₇-C₁₈ alkaryl, C₆-C₁₈ aryl, C₂-C₁₈ alkoxycarbonyl, and C₂-C₁₈ alkaminocarbonyl substituted with a heteroatom within, or attached to, the carbon backbone; R²⁴ is selected from the group consisting of hydrogen and C₁-C₆ alkyl groups; R²⁵ is a linking group; X¹ is selected from the group consisting of carboxyl activating groups, hydrogen, M¹_{1/d}^{d+} and carboxyl protecting groups, wherein M¹ is a metal ion and d is an integer of 1 or 2; R²⁶ is selected from the group consisting of C₁-C₁₈ alkylene, C₂-C₁₈ alkenylene, C₇-C₁₈ aralkylene, C₇-C₁₈ alkarylene and C₆-C₁₈ arylene; in the presence of an initiator compound (VIII)
R²⁷(R²⁸)ₙ—Y¹. (IX)
wherein n is an integer of 1 or more and Y¹ is a radical initiating group, R²⁷ comprises a group selected from the group consisting of hydrogen, C₁-C₁₈ alkyl, C₂-C₁₈ alkenyl, C₇-C₁₈ aralkyl, C₇-C₁₈ alkaryl and C₆-C₁₈ aryl, C₁-C₁₈ alkoxy, C₂-C₁₈ alkeneyloxy, C₇-C₁₈ aralkoxy, C₇-C₁₈ alkaryloxy, C₆-C₁₈ aryloxy and —O—Y¹; and R²⁸ is selected from the group consisting of C₁-C₁₈ alkylene, C₂-C₁₈ alkenylene, C₇-C₁₈ aralkylene, C₇-C₁₈ alkarylene and C₆-C₁₈ arylene; to produce a block copolymer comprising the unit (X) 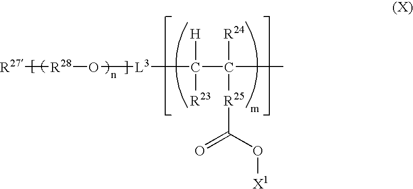
wherein m is an integer of greater than 1 and L³ is a divalent linking group derived from L³; and R²⁷ is R²⁷, or where R²⁷ is Q-Y¹, R²⁷ is 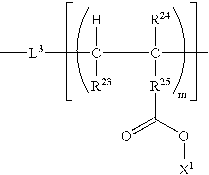
(2) reacting (X) with a reagent HR^{xx}, wherein R^{xx} is selected from the group consisting of NR²⁹R³⁰, SR³¹ and OR³², wherein R²⁹ is a linker group, preferably a peptidic group; R³⁰ is selected from hydrogen, C₁-C₁₈ alkyl, C₂-C₁₈ alkenyl, C₇-C₁₈ aralkyl, C₇-C₁₈ alkaryl, C₆-C₁₈ aryl; R³¹ and R³² are individually selected from the group consisting of hydrogen, C₁-C₁₂ alkyl, C₁-C₁₂ alkenyl, C₁-C₁₂ aralkyl, C₁-C₁₂ alkaryl, C₁-C₁₂ alkoxy and C₁-C₁₂ hydroxyalkyl, and may contain one or more cleavable bonds, to form a derivatised block copolymer having the structure (XI) 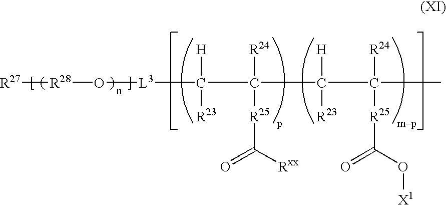
wherein 1≦p≦m.

Y¹ preferably comprises a halogen substituted C₁-C₁₈ alkyl or C₆-C₁₈ aryl group, preferably bromine or chlorine substituted. Preferably Y¹ comprises a group selected from the group consisting of C₁-C₆ alkyl, C₆-C₁₂ aryl, C₁-C₁₂ oxyalkyl and C₁-C₆ acyl substituted with 1 or more halogen atoms. Where Y¹ comprises an alkyl group, it can be branched, linear or cyclical, substituted or unsubstituted with one or more alkyl groups, and is preferably methyl, ethyl, propyl, ^{tert}butyl, ^{sec}butyl, hexyl or octyl. Where Y¹ comprises an aryl group, preferably it is benzyl, tolyl or xylyl. Most preferably Y¹ comprises —COR^{y} group, wherein R¹ is defined above.

L³ is preferably derived from Y¹ and is a C₁-C₁₈ alkylene or C₆-C₁₈ arylene group which may be substituted and/or interrupted with 1 or more heteroatoms. Preferably L³ comprises a group selected from the group consisting of C₁-C₆ alkylene, C₆-C₁₂ arylene, C₁-C₁₂ oxyalkylene and is carbonyl-C₁-C₆ alkylene. Where L³ is an alkylene group, it can be branched, linear or cyclical, substituted or unsubstituted with one or more alkyl groups, and is preferably methylene, 1,2-ethylene, 1,2-propylene, 1,3-propylene, ^{tert}butylene, ^{sec}butylene, hexylene or octylene. Where L³ is an arylene group, preferably it is benzylene, tolylene or xylylene. Most preferably L³ comprises a —COR^{a} group, wherein R³ is selected from the group consisting of C₁-C₆ alkylene or C₆-C₁₂ arylene, preferably methylene, 1,2-ethylene, 1,2-propylene, 1,3-propylene, ^{tert}butyl and ^{sec}butyl

The electron withdrawing group X¹ is preferably a carboxylate activating group, and is preferably selected from the group consisting of N-succinimidyl, pentachlorophenyl, pentafluorophenyl, para-nitrophenyl, dinitrophenyl, N-phthalimido, norbornyl, cyanomethyl, N-pyridyl, N-trichlorotriazine, 5-chloroquinilino, and N-imidazole. Preferably X¹ is an N-succinimidyl or imidazole moiety.

R²⁵ is preferably the same as R².

R²⁷ is preferably selected from hydrogen, C₁-C₆ alkyl, C₁-C₆ alkoxy, C₂-C₁₀ alkenyl, C₇-C₁₀ aralkyl, C₇-C₁₀ alkaryl and C₆-C₁₀ aryl and —O—Y¹.

Preferably R²³ is selected from the group consisting of C₁-C₈ alkylene groups and C₆-C₁₂ arylene groups, most preferably methylene, ethylene, propylene and isopropylene.

Preferably HR^{xx} is HR^{xx} as defined above.

Preferably step (1) process is a controlled radical polymerization and (2) is a nucleophillic substitution reaction.

### DETAILED DESCRIPTION OF THE INVENTION

The present invention preferably provides a block copolymer having a polydispersity of less than 1.4, preferably less than 1.2. The block copolymer is preferably an activated polyacrylate ester that is prepared by Controlled Radical Polymerization. These block copolymers are designed to be derivitisable and may be used to form polymer-drug conjugates having improved biological profile.

The utility of the invention is that conjugation of a bioactive agent can be prepared in defined reagions of a polymer rather than randomly along the mainchain. The use of narrow molecular weight polymer precursor allows more efficient preclinical development to understand the range of aqueous solution based structure-property correlations that can be exploited to optimise the biological profile of polymer-drug conjugates.

The utilisation of the co-blocked polymeric precursors will allow for the preparation of water soluble, narrow MWD functionalised excipients that can be derived copolymers of poly(ethylene glycol) polyacrylic- and methacrylic acids that are further functionalised on the non-PEG block.

A particularly preferred block copolymer of the present invention comprises the structure (XII) 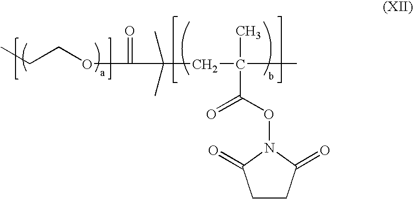
wherein a and b are integers of 1 or more, and preferably define the blocks of the A-B type block copolymer. The activating moiety is an N-succinimidyl group. This particular group has been found to be particularly stable in solution and resists spontaneous hydrolysis. This block copolymer may be produced by Atom Transfer Polymerization using a Cu(I)Br(pentamethyldiethylene) mediator. The polymerization involved the reaction of a monomer (XIII) with a polyethyleneglycol initiator compound (XIV) in a suitable aprotic solvent.

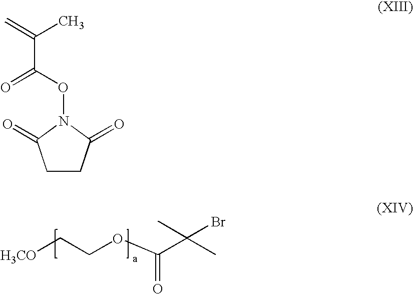

In one preferred embodiment the solvent is tetrahydrofuran. In another preferred embodiment the solvent is dimethylsulphoxide and optionally dimethylformamide in admixture thereof. A further particularly preferred embodiment uses ethylene carbonate as solvent. The reaction is preferably carried out under a nitrogen atmosphere and at a temperature of 0-150° C. A preferred temperature range is 30-80° C., most preferably 50-70° C.

The block copolymer comprising the unit (XII) may subsequently be derivatised. The carboxyl activating group may be substituted by a suitable nucleophilic reagent. In order to form polymer drug conjugates it is preferable to derivatise unit (XII) with a pendant moiety. Such a moiety could comprise a aminoacyl linker or a hydrolytically labile linker as defined hereinbefore. Such a linker can degrade when entering the lysosome of a diseased cell, thus releasing a drug or drug precursor directly to the target site.

Preferably a pendent moiety comprises a Gly-Leu-Phe-Gly linker or a cis aconityl linker. Such a pendent linker may be covalently attached to a drug prior to block copolymer derivitisation or may be capable of being derivatised subsequent of attachment of the pendent moiety to the block copolymer backbone.

In a preferred embodiment the block copolymer comprising the unit (XII) is reacted with less than 1 equivalent of a pendent group, thus only substituting a pre-specified number of N-succinimidyl moieties. This allows a second, quenching step, which substitutes the remaining N-succinimidyl groups with a solubilising group. Such a group aids in the solubilisation of the block copolymer in aqueous solutions such as biological fluids. A preferred quenching agent should comprise a hydrophillic amine or amino acid, preferably a hydroxylated amine, for example 2-hydroxypropylamine. Amine terminated PEG may also be used. Alternatively, the carboxyl activating group may be hydrolysed to produce a free carboxylic acid moiety. An overview of a preferred reaction process is provided in scheme 1 below. In this particular example, the drug doxorubicin is attached to the block copolymer via a GLFG linker.

Preferably, a number of different bioactive agents may be conjugated to the polymer chain.

a and b are integers in the range of 1 to 500 and c is the number equivalent of pendent moieties reacted with the activated block copolymer. CRP processes are known to result in the presence of dormant initiating moieties at the chain ends of linear polymers.

The present invention is also concerned with the use of the block copolymers described above to prepare physiologically soluble polymer bioactive agent conjugates, polymer therapeutics, functionalised polymers, pharmaceutical compositions and materials.

Utilising the block precursor (XII) more controlled placement of the bioactive conjugating pendent chains along the polymer mainchain is possible because this conjugation will only occur in the block with the N-succinimidyl groups. This will result in co-block copolymers, comprising hydrophilic and hydrophobic blocks, to form polymeric micelles in solution. A significant advantage of these systems is the ability to design higher molecular weight micellar aggregates that will display prolonged circulation times that can maximise tumour capture by the EPR effect. Upon micelle disassociation, the individual block copolymer molecules are safely excreted, and as long as they are of low enough molecular weight these polymers can be non-biodegradable. Additionally poly(acrylic acid), poly(methaacrylic acid) and poly(ethylene glycol) based excipients are widely used to modify adhesion, swelling and pH dependent properties of tablets and pharmaceutical formulations. The utilisation of the co-blocked polymeric precursor (XI) allows for the preparation of water soluble, narrow molecular weight distribution functionalised excipients derived copolymers of poly(ethylene glycol)polyacrylic- and methacrylic acids that are further functionalised on the non-PEG block. Since coblock polymers form aggregated micellar structures these new functionalised excipient may be potentially developed into novel formulations for the oral administration of bioactive agents.

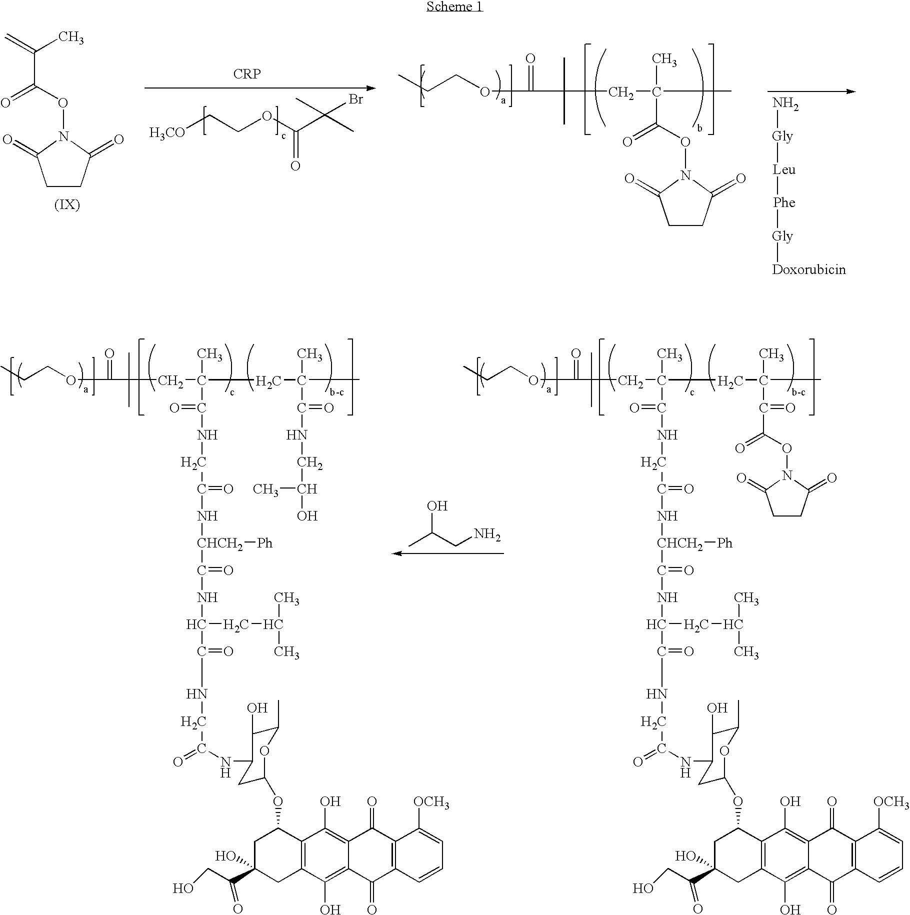

### EXAMPLE 1

Preparation of Macroinitiators 2.

The PEG (polyethylene glycol) macroinitiators were prepared by the procedure of Jankova et al (Macromolecules (1998), 31, 538-541). Triethylamine (12.5×10⁻³ mol, 1.265 g, 1.75 ml) in 15 ml dry CH₂Cl₂ was added to a 250 ml three-neck round-bottom flask equipped with a condenser, dropping funnel, gas inlet and a magnetic stirrer. After cooling to 0° C. 2,2-bromoisobutyryl bromide (12.5×10⁻³ mol, 2,874 g, 1.55 ml) in 10 ml CH₂Cl₂ was added and the mixture purged with nitrogen. Then monomethoxy capped PEG (Mn=2,000 g/mol) (5×10³ mol, 10 g) in 50 ml CH₂Cl₂ was added dropwise during 1 h under nitrogen. The PEG had been previously dried by azeotropic distillation in toluene and the residual toluene removed in vacuum. The temperature of the reaction mixture was allowed to rise to room temperature and the reaction continued for 18 h. The solution was filtered, half of the solvent evaporated under vacuum and the product was precipitated in cold ether. The precipitate was recrystallised in absolute ethanol (stored overnight in the fridge). The macroinitiator was filtered, washed with cold ether and dried under vacuum. The crude product was purified by dissolving 4 g in 80 ml water. The solution pH was raised to pH 8 in order to hydrolyse the excess of i-BuBr. Then the solution was extracted with CH₂Cl₂ (70 ml). A stable emulsion was obtained and several hours were needed for complete phase separation. The solvent was removed in vacuum. The product was dissolved in hot EtOH and put in a fridge to crystallise. Then it was filtered and washed with ether and dried under vacuum. The purified product was white in colour. The degree of substitution calculated by the H MNR spectra.

This procedure was also used to prepare monomethoxy capped PEG macroinitiators derived from PEG 5000 and PEG 10000.

### EXAMPLE 2

Preparation of Co-Block, Narrow Molecular Weight Polymer Precursors 3.

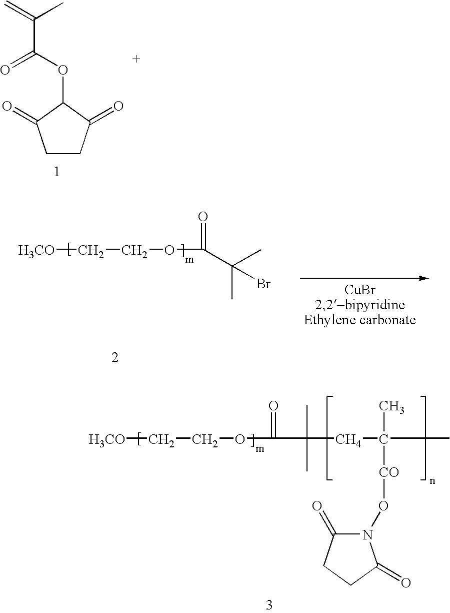

General polymerisation quantisations (reagents and conditions) are outlined in table 1

A mixture of monomer 1 (as synthesised in WO 01/18080), ethylene carbonate, and bipyridine was placed in a tube sealed with septum and it was purged with argon for 5 min and then the CuBr was added. The mixture was gently heated to form a solution (deep brown in colour) and purged with argon for another 30 min. Then a solution of the PEG macroinitiator 2 in the amount relative to the monomer specified in the table in ethylene carbonate (gently heated to melt both the ethylene carbonate and 2) was purged with argon for 10 min and added to the monomer solution by syringe washed with argon. The mixture was placed in a oil bath and stirred. The reaction was stopped by exposure to air, cooling and diluting with DMF. Then the solution was passed through a column filled with alumina and the polymer precipitated in MeOH. The precipitate was filtered, washed with ether and dried in vacuum. The product was obtained as white powder.

TABLE 1
shows Polymerisation conditions and yield and molecular
weight characteristics of polymerisations conducted with macroinitiator 2
derived from PEG of molecular weight 2000 g/mol.

| | 1 | 2 | CuBr | Bpy | 1:2:CuBr: | EC¹ | T | Time | Yield | | PD |
|---|---|---|---|---|---|---|---|---|---|---|---|
| | mmol (g) | (mmol) (g) | (mmol) (g) | (mmol) (g) | bpy | (g) | (° C.) | (h) | (%) | Mn² | (Mw/Mn) |
| 1 | 5 | 0.1 | 0.1 | 0.2 | 50:1:1:2 | 1.3 | 80 | 1.5 | 56 | 17,000 | 1.39 |
| | 0.917 | 0.215 | 0.0157 | 0.0342 | | | | | | | |
| 2 | 5 | 0.1 | 0.1 | 0.2 | 50:1:1:2 | 1.0 | 80 | 1.0 | 40 | 17,600 | 1.30 |
| | 0.917 | 0.215 | 0.0157 | 0.0342 | | | | | | | |
| 3 | 5 | 0.1 | 0.1 | 0.2 | 50:1:1:2 | 1.3 | 80 | 5.0 | 87 | 20,100 | 1.53 |
| | 0.917 | 0.215 | 0.0157 | 0.0342 | | | | | | | |
| 4 | 5 | 0.05 | 0.05 | 0.1 | 100:1:1:2 | 0.65 | 80 | 5.0 | 100 | 31,300 | 1.32 |
| | 0.917 | 0.1075 | 0.0078 | 0.0171 | | | | | | | |
| 5 | 2.5 | 0.05 | 0.05 | 0.1 | 50:1:1:2 | 0.65 | 60 | 5.0 | 20 | 13,230 | 1.35 |
| | 0.458 | 0.1075 | 0.008 | 0.0171 | | | | | | | |
| 6 | 5 | 0.1 | 0.1 | 0.2 | 50:1:1:2 | 1.3 | 80 | 5.5 | 32 | 19,700 | 1.29 |
| | 0.917 | 0.215 | 0.0157 | 0.0342 | | | | | | | |
| 7 | 2.5 | 0.05 | 0.05 | 0.1 | 50:1:1:2 | 0.65 | 100 | 5.0 | 64 | 28,000 | 1.38 |
| | 0.458 | 0.1075 | 0.005 | 0.0171 | | | | | | | |
| 8 | 2.5 | 0.5 | 0.05 | 0.1 | 50:1:1:2 | 0.65 | 80 | 5.0 | 56 | 25,700 | 1.29 |
| | 0.458 | 0.1075 | 0.008 | 0.0171 | | | | | | | |
| 9 | 2.5 | 0.05 | 0.05 | 0.1 | 50:1:1:2 | 0.65 | 80 | 5.0 | 47 | 22,800 | 1.27 |
| | 0.458 | 0.1075 | 0.008 | 0.0171 | | | | | | | |
| 10 | 2.5 | 0.05 | 0.05 | 0.1 | 50:1:1:2 | 0.5 | 80 | 5.0 | 743 | 30,200 | 1.28 |
| | 0.458 | 0.1075 | 0.008 | 0.017 | | | | | | | |
| 11 | 2.5 | 0.05 | 0.025 | 0.15 | 50:1:0.5:3 | 0.5 | 80 | 3.0 | 42.4³ | 18,100 | 1.25 |
| | 0.458 | 0.1075 | 0.004 | 0.017 | | | | | | | |
| 12 | 5 | 0.05 | 0.05 | 0.1 | 100:1:1:2 | 0.5 | 110 | 4:35 | 100 | 18,600 | 1.33 |
| | 0.917 | 0.1075 | 0.008 | 0.0171 | | | | | | | |

¹EC = ethylene carbonate
²Gel permeation chromatography used DMF eluent with PMMA standards
³The reaction mixture was purged with argon for 1 hour.

### EXAMPLE 3

Conjugation reactions are included to demonstrate utility of the precursor to make functionalised polymers in narrow molecular weight distribution.

Conjugation Reactions of Polymer Precursor 3.

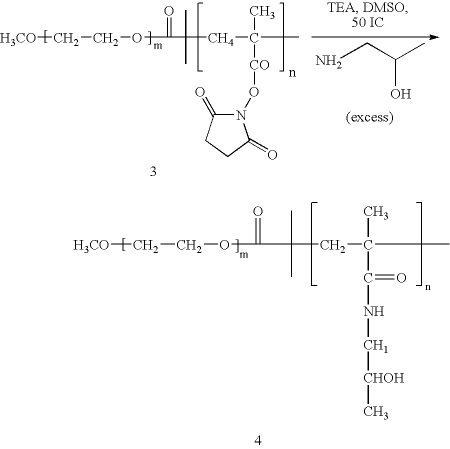

The co-blocked precursor 3 (0.1 g; Mn=32,500 g/mol as determined by GPC with DMF eluent) was dissolved in anhydrous DMSO (0.3 g) and purged with argon for 15 min. 1-Amino-2-propanol (0.2 g) was dissolved in anhydrous DMSO (0.1 g) and purged with argon for 15 min, then the vial equipped with stirrer, was placed in an oil bath at 50° C. The polymer solution was added dropwise (for ˜15 min) by a syringe. The reaction mixture was allowed to react for 1.5 h. Then the product was precipitated in acetone:ether (1:1). The water soluble co-block polymer 4 was dissolved in MeOH and re-precipitated in acetone:ether=(1:1). The IR spectrum displayed no absorption at 1732 cm¹ indicating all the N-hydroxysuccinimide had been displaced. GPC analysis indicated the Mn was 25,500 g/mol (PD=1.35) demonstrating that the hydrodynamic radius of polymer 4 differed significantly from the starting precursor polymer 3. H-NMR analysis of product 4 demonstrated the PEG block was covalently bound.

### EXAMPLE 4

Conjugation of 10 mole percent peptide drug model 5 followed by conjugation of 1-amino-2-propanol to give coblocked conjugate 7.

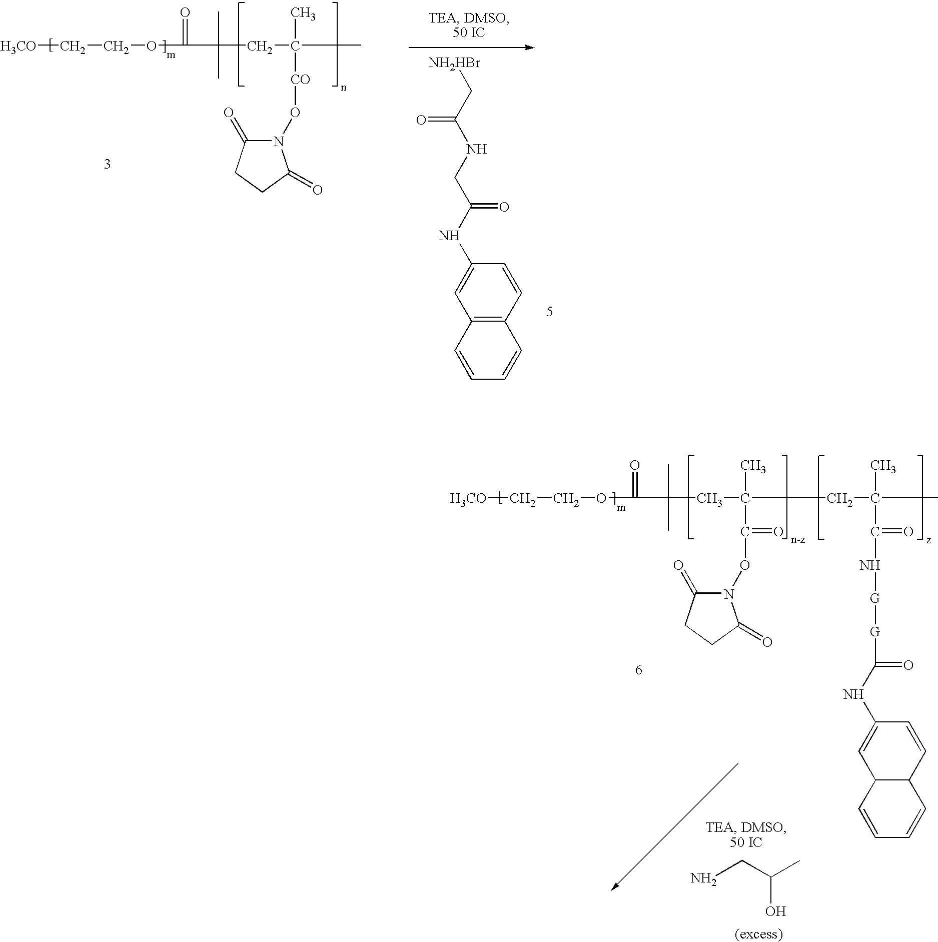

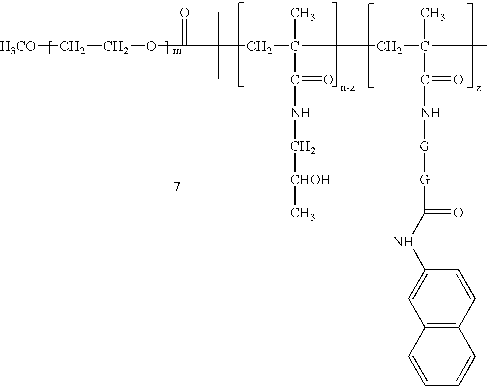

The co-blocked precursor 3 (0.15 g; Mn=19,700 g/mol as determined by GPC with DMF eluent) was dissolved in anhydrous DMSO (1.25 ml) and purged with argon for 10 min. Since the macroinitiator 2 was found to have a GPC molecular weight Mn=4,400 g/mol₁ the amount of reactive units in this sample of precursor polymer 3 was calculated to be 6.3×10⁻⁴ mol. To conjugate 10 mole percent 5, 6.3×10⁻⁵ mol (0.0229) of 5 was added to the reaction mixture and the flask placed in an oil bath at 50° C. A solution of triethylamine (12.6×10⁻⁴ mol, 0.0127 g) in 0.25 ml DMSO (previously purged with Ar) was added drop-wise over 1-2 min. The mixture was stirred 10 min and then the solution of 1-amino-2-propanol (12.6×10⁻⁴ mol=0.0946 g) in 0.5 ml DMSO was added drop-wise for 10 min and the stirring continued for additional 50 min at 50° C. The IR spectrum displayed no absorption at 1732 cm⁻¹ indicating all the N-hydroxysuccinimide had been displaced and the conjugate was isolated in cold THF after centrifugation. H-NMR analysis of product 7 demonstrated the PEG block was covalently bound.

1. A block copolymer comprising the unit (I) 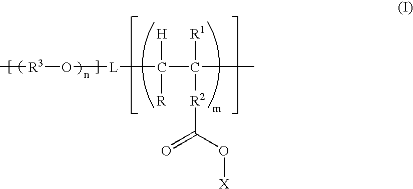
wherein:
R is selected from the group consisting of hydrogen, C₁-C₁₈ alkyl, C₂-C₁₈ alkenyl, C₇-C₁₈ aralkyl, C₇-C₁₈ alkaryl, C₆-C₁₈ aryl, carboxylic acid, C₂-C₁₈ alkoxycarbonyl, C₂-C₁₈ alkaminocarbonyl, or any one of C₁-C₁₈alkyl, C₂-C₁₈ alkenyl, C₇-C₁₈ aralkyl, C₇-C₁₈ alkaryl, C₆-C₁₈ aryl, C₂-C₁₈ alkoxycarbonyl and C₂-C₁₈ alkaminocarbonyl substituted with a heteroatom within, or attached to, the carbon backbone;
R¹ is selected from the group consisting of hydrogen and C₁-C₆ alkyl groups;
R² is a linking group;
X is an electron withdrawing group;
R³ is selected from the group consisting of C₁-C₁₈ alkylene, C₂-C₁₈ alkenylene, C₇-C₁₈ aralkylene, C₇-C₁₈ alkarylene and C₆-C₁₈ arylene;
L is a divalent linker joining the blocks;
and m and n are each an integer of greater than 1.

2-37. (Cancelled)

38. The block copolymer according to claim 1 in which m and n are integers of 5 to 300.

39. The block copolymer according to claim 1 which has a polydispersity of less than 1.4 and a molecular weight (Mw) of less than 100,000.

40. The block copolymer according to claim 1 which is water soluble.

41. The block copolymer according to claim 1 in which X is a carboxylate activating group selected from the group consisting of N-succinimidyl, pentachlorophenyl, pentafluorophenyl, para-nitrophenyl, dinitrophenyl, N-phthalimido, norbornyl, cyanomethyl, N-pyridyl, N-trichlorotriazine, 5-chloroquinilino, and N-imidazole.

42. The block copolymer according to claim 1 in which R is selected from the group consisting of hydrogen, C₁-C₆ alkyl, C₁-C₆ alkenyl, C₁-C₆ aralkyl and C₁-C₆ alkaryl, C₂-C₈ alkoxycarbonyl, and C₂-C₈ alkaminocarbonyl.

43. The block copolymer according to claim 1 in which R¹ comprises a moiety selected from the group consisting of hydrogen, methyl, ethyl, propyl, butyl, and pentyl or isomers thereof.

44. The block copolymer according to claim 1 in which R² is a bond or a divalent group selected from a carbonyl, C1-C18 alkylene and/or C6-C18 arylene group which may be substituted with 1 or more heteroatoms.

45. The block copolymer according to claim 44 in which R² is selected from the group consisting of C1-C6 alkylene, C6-C12 arylene, C1-C12 oxyalkylene and carbonyl-C1-C₆ alkylene.

46. The block copolymer according to claim 45 in which R² is selected from the group consisting of methylene, 1,2-ethylene, 1,3-propylene, hexylen, octylene, benzylen, tolylene and xylylene.

47. The block copolymer according to claim 1 in which the R³ groups, which may be all the same or different from one another, comprise C1-C8 alkylene groups.

48. The block copolymer according to claim 47 in which all R³ groups are the same and comprise a moiety selected from the group consisting of 1,2-ethylene and 1,2-propylene.

49. The block copolymer according to claim 1 in which L comprises a C1-C18 alkylene or C6-C18 arylene group which may be substituted and/or interrupted with 1 or more heteroatoms.

50. The block copolymer according to claim 49 in which L is selected from the group consisting of C1-C6 alkylene, C6-C12 arylene, C1-C12 oxyalkylene and C1-C6 acyl.

51. The block copolymer according to claim 50 in which L comprises a —COR^{a} group, wherein R^{a} is selected from the group consisting of C1-C6 alkylene and C8-C12 arylene.

52. The block copolymer according to claim 1 which comprises the structure (II) 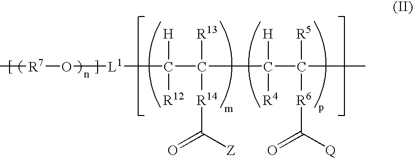
wherein:
R⁴ is selected from the group consisting of hydrogen, C1-C18 alkyl, C2-C18 alkenyl, C7-C18 aralkyl, C7-C18 alkaryl, C6-C18 aryl, carboxylic acid, C2-C18 alkoxycarbonyl, C2-C18 alkaminocarbonyl, or any one of C1-C18 alkyl, C2-C18 alkenyl, C7-C18 aralkyl, C7-C18 alkaryl, C6-C18 aryl, C2-C18 alkoxycarbonyl, and C2-C18 alkaminocarbonyl substituted with a heteroatom within, or attached to, the carbon backbone;
R⁵ is selected from the group consisting of hydrogen and C1-C6 alkyl groups;
R⁶ is a linking group;
Q is a solubilising group selected from the group consisting of hydroxyl, C1-C12 alkyl, C2-C12 alkenyl, C7-C12 aralkyl, C7-C12 alkaryl, C1-C12 alkoxy, CI—C₁₋₂ hydroxyalkyl, C1-C12 alkylamino, C1-C12 hydroxyalkylamino, or any one of C1-C12 alkyl, C2-C12 alkenyl, C7-C12 aralkyl, C7-C12 alkaryl, C1-C₁₂ alkoxy, C₁-C12 hydroxyalkyl, C1-C12 alkylamino, C1-C12 alkylamino substituted with an amine, hydroxyl, carbonyl or thiol group;
R⁷ is selected from the group consisting of C1-C18 alkylene, C2-C18 alkenylene, C7-C18 arakylene, C7-C18 alkarylene and C6-C18 arylene;
n, m and p are each an integer of greater than 1;
R¹² is selected from the group consisting of hydrogen, C1-C18 alkyl, C2-C18 alkenyl, C7-C18 aralkyl, C7-C8 alkaryl, C6-C18 aryl, carboxylic acid, C2-C18 alkoxycarbonyl, C2-C18 alkaminocarbonyl, or any one of C1-C18 alkyl, C2-C18 alkenyl, C7-C18 aralkyl, C7-C18 alkaryl, C6-C18 aryl, C2-C18 alkoxycarbonyl, and C2-C18 alkaminocarbonyl substituted with a heteroatom within, or attached to, the carbon backbone;
R¹³ is selected from the group consisting of hydrogen and C1-C6 alkyl groups;
R¹⁴ is a linking group;
L¹ is a divalent linker joining the blocks;
Z is a pendent group selected from the group consisting of OM_{1/d}^{d+}, NR⁸R⁹, SR¹⁰, OR¹¹ and OX, wherein X is defined above, M is a metal ion and d is an integer of 1 or 2, R⁸ comprises an alkyl group, preferably an aminoacyl substituted alkyl group, more preferably oligopeptidy group;
R⁹ is selected from hydrogen, C1-C18 alkyl, C2-C18 alkenyl, C7-C18 aralkyl, C7-C18 alkaryl;
R¹⁰ and each R¹¹ comprise a group which is individually selected from the group consisting of hydrogen, C1-C12 alkyl, C2-C12 alkenyl, C7-C12 aralkyl, C7-C12 alkaryl and C1-C12 hydroxyalkyl, and may contain one or more cleavable bonds and may comprise a bioactive agent.

53. The block copolymer according to claim 52 in which Z comprises one or more aminoacyl groups.

54. The block copolymer according to claim 53 in which Z comprises a glycine-leucine-phenylalanine-glycine linker.

55. The block copolymer according to claim 52 in which Z comprises a cis-aconityl group.

56. The block copolymer according to claim 52 in which Z comprises a bioactive agent or linker.

57. The block copolymer according to claim 56 in which the bioactive agent is an anti-cancer agent.

58. The block copolymer according to claim 52 in which Q comprises an amine group attached to the R⁶CO carbonyl carbon.

59. A process for the production of a block copolymer, comprising the polymerisation of ethylenically unsaturated monomers including a compound (III) 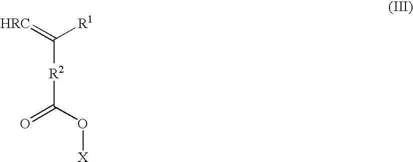
wherein R is selected from the group consisting of hydrogen, C1-C18 alkyl, C2-C18 alkenyl, C7-C18 aralkyl, C7-C18 alkaryl, C6-C18 aryl, carboxylic acid, C2-C18 alkoxycarbonyl, C2-C18 alkaminocarbonyl, or any one of C1-C18 alkyl, C2-C18 alkenyl, C7-C18 aralkyl, C7-C18 alkaryl, C6-C18 aryl, C2-C18 alkoxycarbonyl, and C₂-C18 alkaminocarbonyl substituted with a heteroatom within, or attached to, the carbon backbone;
R¹ is selected from the group consisting of hydrogen and C1-C6 alkyl groups;
R² is a linking group;
X is an electron withdrawing group;
in the presence of an initiator compound (IV)
R¹⁵(R³O)ₙ—Y (IV)
wherein n is an integer of 1 or more and Y is a radical initiating group;
R³ is selected from the group consisting of C1-C18 alkylene, C2-C18 alkenylene, C7-C18 aralkylene, C7-C18 alkarylene and C6-C18 arylene;
R¹⁵ comprises a group selected from the group consisting of hydrogen, C1-C18 alkyl, C2-C18 alkenyl, C7-C18 aralkyl, C7-C18 alkaryl and C6-C18 aryl, C1-C18 alkoxy, C2-C18 alkeneyloxy, C7-C18 aralkoxy, C7-C18 alkaryloxy, C6-C18 aryloxy and —O—Y;
to produce a block copolymer comprising the unit (V) 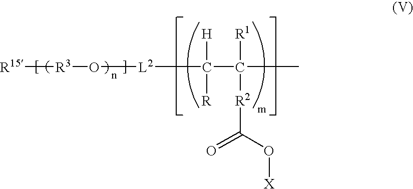
wherein m and n are as defined above and L² is a divalent linking group derived from Y and R^{15′} is R¹⁵, or where R15 is —O—Y, R¹⁵ is

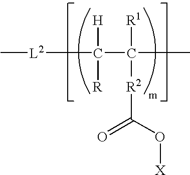

60. The process according to claim 59 in which the groups X, R, R¹, R² and R³ are as defined as follows:
R is selected from the group consisting of hydrogen, C₁-C₁₈ alkyl, C₂-C₁₈ alkenyl, C₇-C₁₈ aralkyl, C₇-C₁₈ alkaryl, C₆-C₁₈ aryl, carboxylic acid, C₂-C₁₈ alkoxycarbonyl, C₂-C₁₈ alkaminocarbonyl, or any one of C₆-C₁₈alkyl, C₂-C₁₈ alkenyl, C₇-C₁₈ aralkyl, C₇-C₁₈ alkaryl, C₆-C₁₈ aryl, C₂-C₁₈ alkoxycarbonyl and C₂-C₁₈ alkaminocarbonyl substituted with a heteroatom within, or attached to, the carbon backbone;
R¹ is selected from the group consisting of hydrogen and C₁-C₆ alkyl groups;
R² is a linking group;
X is an electron withdrawing group;
R³ is selected from the group consisting of C-C₁₋₈ alkylene, C₂-C₁₈ alkenylene, C₇-C₁₈ aralkylene, C₇-C₁₈ alkarylene and C₆-C₁₈ arylene;
and wherein the block copolymer has a polydispersity of less than 1.4 and a molecular weight (Mw) of less than 100,000.

61. The process according to claim 59 in which Y is a —COR^{y} group, wherein R^{y} is selected from the group consisting of halogen substituted C₁-C₆ alkyl or C6-C12 aryl.

62. The process according to any of claim 59 in which L² is selected from the group consisting of C₁-C₆ alkylene, C₆-C₁₂ arylene, Cl-C 12 oxyalkylene and carbonyl-C1-C6 alkylene, wherein R^{a} is selected from the group consisting of C1—C6 alkylene and C6-C₁₋₂ arylene.

63. The process according to claim 59 in which R¹⁵ is selected from hydrogen C1-C6 alkyl, C1-C6 alkoxy, C2-C10 alkenyl, C7-C10 aralkyl, C7-C10 alkaryl and C₆-C₁₀ aryl and —O—Y.

64. The process according to claim 59 which is a controlled radical polymerisation process.

65. The process according to claim 59 in which comonomers are copolymerised with the monomer of the formula III.

66. The process according to claim 59 in which the block copolymer of the formula V is reacted further with a reagent HR^{x}, wherein
R^{x} is selected from the group consisting of NR¹⁹, R²⁰, SR²¹ and OR²², wherein R¹⁹ comprises a linker group;
R²⁰ is selected from hydrogen, C1-C18 alkyl, C₂-C₁₈ alkenyl, C₇-C₁₈ aralkyl, C₇-C₁₈ alkaryl, C₆-C₁₈ aryl;
R²¹ and R²² are selected from the group consisting of hydrogen, C₁-C₁₂ alkyl, C1-C12 alkenyl, C1-C12 aralkyl, C1-C12 alkaryl, C1-C12 alkoxy and C1-C12 hydroxyalkyl, any of which may comprise a bioactive agent substituent and/or may contain one or more cleavable bonds, to form a derivatised block-copolymer having the structure (VI) 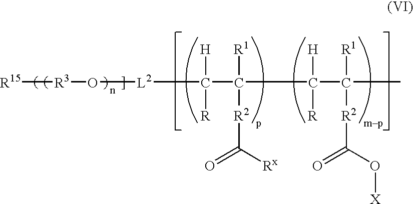
wherein 1≦p>m.

67. The process according to claim 66 in which HRX is H₂NR² and wherein R² comprises an aminoacyl linker or a cis-aconityl linker and a bioactive agent or a ligand.

68. The process according to claim 66 in which the block copolymer of the formula VI is quenched by reacting remaining groups —COOX with an amine group-containing compound.

69. The process for the production of a block copolymer, comprising the steps of polymerising ethylenically unsaturated monomers comprising a compound (VII) 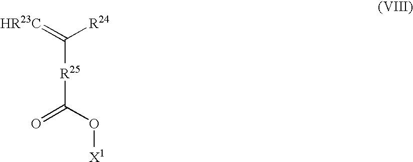
wherein R²³ is selected from the group consisting of hydrogen, C1-C18 alkyl, C2-C18 alkenyl, C7-C18 aralkyl, C7-C18 alkaryl, C6-C18 aryl, carboxylic acid, C2-C18 alkoxycarbonyl, C2-C18 alkaminocarbonyl, or any one of C1-C18 alkyl, C2-C18 alkenyl, C7-C18 aralkyl, C7-C18 alkaryl, C6-C18 aryl, C2-C18 alkoxycarbonyl, and C2-C18 alkaminocarbonyl substituted with a heteroatom within, or attached to, the carbon backbone;
R24 is selected from the group consisting of hydrogen and C1-C6 alkyl groups;
R25 is a linking group;
X¹ is selected from the group consisting of carboxyl activating groups, hydrogen, M_{1/d}^{d+} and carboxyl protecting groups, wherein M¹ is a metal ion and d is an integer of 1 or 2;
R²⁶ is selected from the group consisting of C1-C18 alkylene, C2-C18 alkenylene, C7-C18 aralkylene, C7-C18 alkarylene and C6-C18 arylene; in the presence of an initiator compound (VII)
R²⁷(R²⁸O)ₙ—Y¹ (IX)
wherein n is an integer of 1 or more and Y¹ is a radical initiating group comprises a group selected from the group consisting of hydrogen, C1-C18 alkyl, C2-C18 alkenyl, C7-C18 aralkyl, C7-C₁₈ alkaryl and C6-C₁₈ aryl, C1-C18 alkoxy, C2-C18 alkeneyloxy, C7-C18 aralkoxy, C7-C18 alkaryloxy, C6-C18 aryloxy and —O—Y¹; and
R²⁸ is selected from the group consisting of C1-C18 alkylene, C₂-C₁₈ alkenylene, C7-C18 aralkylene, C7-C18 alkarylene and C6-C18 arylene;
to produce a block copolymer comprising the unit (X) 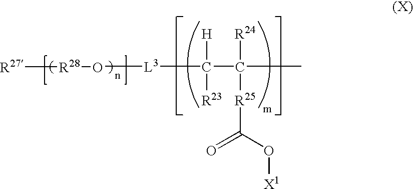
wherein m is an integer of greater than 1 and L³ is a divalent linking group derived from L³; and R^{27′} is R²⁷, or where R²⁷ is —O—Y1, R²⁷ is 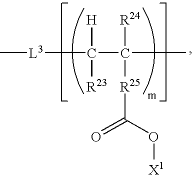
and reacting (X) with a reagent HR^{xx}, wherein R^{xx} is selected from the group consisting of NR²⁹ R³⁰, SR³¹ and OR³², wherein R²⁹ is a linker group;
R³⁰ is selected from hydrogen, C1-C18 alkyl, C2-C18 alkenyl, C7-C18 aralkyl, C7-C18 alkaryl, C6-C18 aryl;
R³¹ and R³² are individually selected from the group consisting hydrogen, C1-C12 alkyl, C1-C12 alkenyl, C1-C12 aralkyl, C₁-C12 alkaryl, C1-C12 alkoxy and C1-C12 hydroxyalkyl, and may contain one or more cleavable bonds, to form a derivatised block copolymer having the structure (XI) 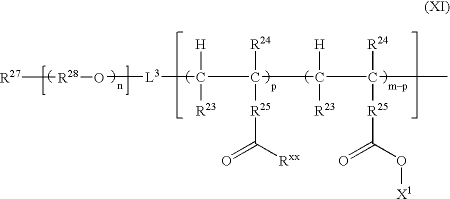
wherein 1≦p≦m.

70. The process according to claim 59 in which the ethylenically unsaturated monomer compound is 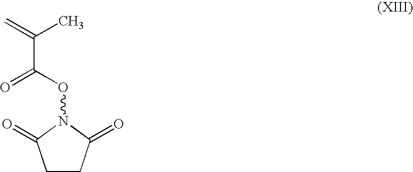
and the initiator is 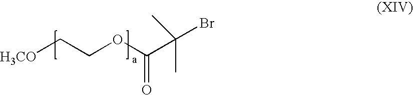
in which a is 1 to 500.

71. The process according to claim 70 in which the copolymer is reacted with a compound H₂N-Gly-Leu-Phe-Gly-Doxorubicin
and in which the product is reacted with 2-hydroxy-propylamine.

72. A block copolymer according to claim 1 having the structure (XII) 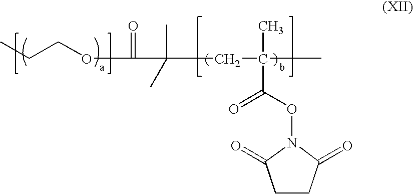
wherein a and b are integers of up to 500.

73. A block copolymer which is obtainable by reacting the block copolymer of claim 36 and a reagent selected to provide a pendant group comprising an aminoacyl linker or a cis-aconityl linker and a bioactive agent.
